(19) United States
(12) Patent Application Publication
(10) Pub. No.: US 2010/0088252 A1
(43) Pub. Date: Apr. 8, 2010

(54) METHODS OF PROVIDING LONG-TERM NUTRITION

(76) Inventors: Hervé Le-Henand, Saint Germain en Laye (FR); Catherine Bailly, Lausanne (CH); Michael Jedwab, Lausanne (CH); Bruce McConnell, Vevey (CH)

Correspondence Address: Nestle HealthCare Nutrition, 12 Vreeland Road, 2nd Floor, Box 697, Florham Park, NJ, 07932, US

(21) Appl. No.: 12/444,940

(22) PCT Filed: Oct. 17, 2007
(86) PCT No.: PCT/EP2007/061112

§ 371 Date: Apr. 9, 2009

Related U.S. Application Data
(60) Provisional application No. 60/862,165, filed on Oct. 19, 2006.

(51) Int. Cl. G06Q 90/00; A61M 5/00

(52) U.S. Cl. 705/500; 604/246

(57) ABSTRACT

A method for providing long-term tube fed nutrition to at least a majority of patients requiring same comprising the steps of providing a line of tube-fed nutrition products that are designed to provide long-term nutritional requirements of at least certain patient populations, the patient population selected from the group consisting of: elderly, maintenance, cancer, digestive pathologies, pediatric, and cystic fibrosis.

### BACKGROUND

The present application relates to nutrition. More specifically, the present invention relates to clinical nutrition.

Due to a variety of diseases, insults, and complications, patients may not be able to obtain the necessary nutrition by ingesting food through the mouth, e.g., eating food. Therefore, it has been known to provide clinical nutrition either enterally or parenterally. A variety of different formulations have been developed to provide such clinical nutrition.

Even with respect to typical enteral nutrition products, these products are designed for short-term use, typically 10 to 14 days. In this regard, the products usually supply the essential nutritional components to provide necessary nutrition to patients having acute pathologies during their hospital stays. Although these products are suitable for such short term use, they have not necessarily been designed for long-term feeding of patients. With advances in medicine resulting in increased life expectancy and better disease treatments, a number of individuals could benefit from products designed to provide long-term enteral nutrition.

### SUMMARY

The present invention provides methods of providing long-term tube fed nutrition to at least a majority of patients requiring same. Furthermore, the present invention relates to methods of providing a business model and business strategy based on fulfilling the needs of long-term enteral nutrition, as well as methods for generating a revenue stream from providing long-term tube fed nutrition to patients.

To this end, the present invention provides a method for providing long-term tube fed nutrition to at least a majority of patients requiring same comprising the step of providing a line of tube fed nutrition products that are designed to provide the long-term nutritional requirements of at least certain patient populations, the patient populations being selected from the group consisting of: elderly, maintenance, cancer, digestive pathologies, pediatric, stroke, coma, respiratory conditions and cystic fibrosis. The method includes marketing each of the product lines to the target patient population.

In an embodiment, the patient receives the product outside of a hospital setting, for example, at home. The nutrition product can be provided to the patient with a feed tube (e.g., nasogastric or percutaneous endoscopic gastrostomy) and other necessary equipment to allow the patient to receive the nutrition product in a non-hospital setting. For example, the long-term nutrition product can be sold as a package with necessary disposables and instructions as to how to administer the nutrition product.

Additionally, the present invention provides a method of providing long-term nutrition to specific patient populations. The method comprising, in an embodiment, the steps of providing at least three distinct long-term tube fed nutrition products and marketing each of the long-term nutrition products to a specific patient for long-term tube fed nutrition. In an embodiment, the patient populations are selected from the group consisting of: maintenance, elderly, cancer, digestive pathologies, pediatric, stroke, coma, respiratory conditions and cystic fibrosis. As noted above, each of the distinct nutrition products can be provided to the patient in a non-hospital setting, e.g., at home, along with disposables such as a feed tube. For example, the nutrition product can be sold as a package with necessary disposables and instructions to administer the product.

Yet further, the present invention provides a business based on providing long-term nutrition to distinct patient populations in a non-hospital setting, e.g., home, comprising the steps of: providing to at least three distinct patient populations three distinct long-term tube fed nutrition products with necessary disposables and instructions to allow the patient to receive long-term tube fed enteral nutrition at home, the patient populations comprising at least maintenance and elderly. In an embodiment, the patient populations additionally comprise pediatric, digestive pathologies, and cancer.

The present invention also provides a method of providing long-term tube fed nutrition to at least three distinct patient populations comprising the steps of: providing three distinct tube-fed nutrition products designed to provide long-term nutrition; providing equipment to allow the patient to receive the long-term nutrition product in a non-hospital setting, e.g., home; and providing training to the patient on how to use the equipment to receive long-term nutrition at home or other non-hospital setting. The patient population can be selected from the group consisting of: maintenance, elderly, cancer, digestive pathologies, and pediatric. In an embodiment, the equipment comprises a feed tube and pump or flow regulator. The training includes an individual that meets with the patient. The individual is typically a healthcare professional such as a doctor, nurse, and dietitian. In an embodiment, the method can include the step of providing the patient with the ability to provide feedback to a prescriber of the long-term nutrition product. If desired, the method can include the steps of delivering the long-term nutrition product and equipment to a home of the patient.

In yet another embodiment of the present invention, a method of providing a complete line of enteral nutrition products is provided that comprises marketing both long-term enteral nutrition products directed to distinct patient populations and standard enteral nutrition products.

An advantage of the present invention is to provide long-term enteral nutrition to patients, in a non-hospital setting if desired.

Another advantage of the present invention is to provide a business formulated, at least in part, on providing long-term enteral nutrition.

Still further, an advantage of the present invention is to provide patients with the ability to receive long-term enteral nutrition designed for specific needs.

Moreover, an advantage of the present invention is to provide a full line of long-term enteral nutrition products.

Furthermore, an advantage of the present invention is to provide improved methods for providing, delivering, and/or generating revenue from clinical nutrition.

Additionally, an advantage of the present invention is to provide a line of enteral nutrition products that meet both short-term and long-term needs as well as the needs of specific patient populations.

Additional features and advantages are described herein, and will be apparent from, the following Detailed Description.

### DETAILED DESCRIPTION

The present invention relates to clinical nutrition. More specifically, the present invention relates to providing long-term tube fed nutrition to patients. As used herein, the term “long-term” means greater than one month (30 days). As used herein, the term “tube fed” means to provide a product to a patient through a feed tube that is received within a portion of the digestive tract of a patient, for example, a nasogastric feed tube or a percutaneous endoscopic gastrostomy tube. As used herein, the term “standard enteral nutrition product” refers to products that are not specifically advertised or promoted for long-term use. A variety of such products are available, for example, from Nestlé, Abbott, Novartis, Numico, and Fresenius.

Pursuant to the present invention, methods are provided for providing long-term tube fed nutrition to patients as well as using such methods to create, develop, or maintain a business based in whole or in part on same. These methods can be part of a business that includes the manufacture, marketing, and/or sale of the product either directly to patients or to third parties such as healthcare facilities or healthcare professionals. The products may be used to provide nutrition to the patients either in hospital, in nursing homes, in day care or other out-patient facilities or in the home of the patient.

The long-term tube fed nutrition products are preferably designed for specific patient populations. These patient populations are unable to consume a normal diet. As used herein, the term “normal diet” means to receive at least substantially all nutrition by eating, i.e., using one's mouth, without the use of any feed tube or parenteral feed.

The specific patient populations to which the present invention is directed include at least in part, inter alia, maintenance, elderly, cancer, pediatric, digestive pathologies, and cystic fibrosis patients. As used herein, “maintenance patient” refers to an adult patient under the age of sixty-five who cannot receive nutrition through a normal diet but who is normo-metabolic (i.e. not suffering from a metabolic disorder). Such a patient may previously have undergone surgery for a cancer of the head or neck leaving an incomplete digestive tract or an inability to swallow, may have received an injury to the neck leaving him or her unable to swallow or may be unable to swallow as a result of neurological damage caused by a stroke for example. As used herein, “elderly patient” refers to a patient similar to maintenance patient, but at least sixty-five years of age. As used herein, “cancer patient” refers to a patient who cannot receive nutrition through a normal diet or is malnourished and who is suffering from an active cancer i.e. is not normo-metabolic. The active cancer may be a cancer of the neck, head, or digestive tract, or it may be a recurrence of cancer in a patient who has previously suffered and been treated for a cancer of the head, neck or digestive tract which has left him or her unable to eat a normal diet. As used herein, “pediatric patients” are those patients one (1) year of age or older that cannot receive nutrition through a normal diet due to disease, disorder, or other disability. As used herein, “digestive pathologies” refers to those patients that cannot receive nutrition through a normal diet due to a digestive tract disease or disorder.

Pursuant to the present invention, methods for providing distinct products that provide long-term tube fed nutrition to specific patient populations are provided. As used herein, “distinct product(s)” refers to a product that is designed and marketed for different patient populations.

The present invention provides methods as well as products that are optimized and/or improved for long-term use. In an embodiment, these product are provided to the patient outside of a hospital setting. For example, the products can be provided in a nursing home, out care patient center, or even the home of the patient. Preferably, the nutrition products are housed in a plastic bag. A variety of such bags are known, for example, 500 ml, 1000 ml, and 1500 ml bags are known in the art. It should be noted, however, that any suitable container can be used to house the nutrition product. Typically, the patient will receive 1500 ml of product a day, though this can vary as will be readily apparent to those skilled in the art.

In an embodiment, some of the products are designed so that they can provide complete long-term nutrition and attempt to mimic what is referred to herein as 5/8 a day. As used herein, the term “5/8 a day” refers to governmental guidelines to consumers to eat five to eight helpings of fruits and vegetable per day. Thus, in an embodiment, some of the products are designed so that, to the extent possible, they attempt to mimic a normal diet that is preferably ingested by individuals that do not require a tube fed product by providing micronutrients and phytonutrients found in fruit and vegetables. In an embodiment, the present invention provides a method of designing long-term enteral nutrition products based on attempting to mimic the 5/8 a day. By providing such a nutrition product, the patient's antioxidant status can be maintained as well as metabolic status. A goal being to place these patients in a state comparable, to the extent possible, to that of a completely healthy individual of the same age eating a balanced diet.

Phytonutrients have been found to provide the following characteristics: antioxidant, anti-inflammatory, detoxification, cancer protective, prevention of atherosclerosis, alleviation of metabolic syndromes, and prevention of bone loss. To achieve the necessary phytonutrients, the compositions of the present invention can include carotenoids such as lycopene (tomato), B-carotene (carrot, spinach, tomato), lutein (spinach), B-cryptoxanthin, vitamins such as mixed tocopherols (oils and nuts), and vitamin C (orange); and polyphenols such as catechins (green tea).

As noted above, pursuant to the present invention, distinct nutrition products are provided that are designed for specific patient populations. These nutrition products are described in detail in patent applications being filed by the Applicants herewith. Applicants hereby incorporate by reference the entire disclosures of U.S. Patent Applications entitled “LONG-TERM TUBE FED NUTRITION FOR MAINTENANCE,” filed herewith, bearing Ser. No. ______, “LONG-TERM TUBE FED NUTRITION FOR ELDERLY,” filed herewith, bearing Ser. No. ______, and “LONG-TERM TUBE FED NUTRITION FOR CANCER,” filed herewith, bearing Ser. No. ______. These products are designed for the following specific patient populations: maintenance; elderly; and cancer. Although the disclosures of each of those patients are incorporated by reference herein, set forth below, as examples, are specific nutrition products designed for the specific patient populations.

Preferably, the products include the necessary nutritional components to provide complete nutrition to the patient on a long-term basis. In this regard, the products include, among other possible ingredients: protein, carbohydrate, fat, fibres, vitamins, minerals, trace elements and phytonutrients. In an embodiment, the products substantially, if not completely, comply with at least certain governmental requirements. As used herein, “governmental requirements” means any recommendations from any one of the following governments: U.S., typically the USRDA, German, typically the German RDA, and French, typically the French RDA. In an embodiment, the nutrition product meets or exceeds at least one of the governmental requirements.

Pursuant to the present invention, nutrition products can be provided to the patient with the necessary equipment, including disposables, to utilize the nutrition product. In this regard, the nutrition product can be provided with a feeding tube, for example, a nasogastric feed tube or percutaneous endoscopic gastrostomy tube, means for controlling the feed of the product, for example, a feed pump or flow regulator for a gravity feed of the product, and instructions. Thus, the nutrition products, pursuant to the method, can be provided with all necessary equipment so that the nutrition product can be administered in any setting, e.g., at home. Typically, the feed tube will be supplied along with the nutrition product to the patient. With respect to the pump or flow regulator, it can either be supplied to the patient or center in which the patient is receiving the product, leased, or provided as a loaner.

Preferably, the nutrition product is provided with instructions as to its use. These instructions can be provided so that they instruct either the patient or the healthcare provider as to how to use the respective nutrition products. The instructions can be provided in written form (as a document), audio form (e.g., tape or CD), and/or visual form (e.g., videotape or DVD). In an embodiment, the instructions will include the amount of nutrition product to be ingested on a daily basis. However, the instructions can also include how to use, for example, the feed tube as well as the pump or flow regulator. Either the healthcare provider, patient, or other individual can use the instructions. In addition, separate training can be provided to, for example, the healthcare provider or patient. In an embodiment, the method includes providing individuals that are skilled in training patients on how to use the nutrition product and equipment.

In an embodiment, the present invention includes a method of providing the products directly to the patient. For example, the method can include providing the nutrition product to the patient at his or her home. However, the product can also be provided to the hospital, or some other facility such as a nursing home or an outpatient healthcare center.

Pursuant to an embodiment, the method of the present invention includes the interface of a healthcare practitioner with the patient. In this regard, in an embodiment, the method of the present invention includes having a healthcare practitioner instruct the patient as to how to use the product. The healthcare practitioner can include, by way of example and not limitation, a doctor, a nurse, a paramedic, or a medical assistant. The interface also allows feedback from the patient to the healthcare practitioner and ultimately the manufacturer and/or distributor of the product. This allows the manufacturer to improve or modify the product or other services. Moreover, the feedback allows the supplier/healthcare practitioner to monitor how easy the equipment is to use as well as compliance to ensure the patient is receiving sufficient feed.

In an embodiment, the present invention provides a business or business model based on either a partial or full-service enteral nutrition business. In this regard, a range of long-term enteral nutrition products directed to distinct patient populations can be provided. These products may or may not be marketed with standard enteral nutrition products and, if desired, parenteral nutrition products. By way of example, some standard enteral nutrition products sold by Nestlé include Nutren® 1.0, Nutren® 1.5, Nutren® Fiber, Sondalis® 1.5, and Sondalis® HP. The nutrition products can, in addition to being sold or marketed for a hospital setting, be sold or marketed for out-of-hospital use, e.g., home, nursing home, etc. The business can supply the nutrition product along with necessary equipment. The service provided by the business may also include providing necessary healthcare practitioners. The healthcare practitioners can not only assist the patient but can help with the patient feedback, allowing improvements to the delivery system, use, and/or product. Further, such interface can be used to develop relevant databases. If desired, the enteral nutrition product can be provided to the patient with the help of a third party such as a hospital, nursing home, home healthcare center, etc.

Pursuant to the present invention, a business model can be provided under which the long-term nutritional needs of a majority of tube-fed patients can be met. In this regard, specific patient populations can be provided with necessary tube-fed nutrition products that will provide long-term nutrition to the patients. Pursuant to the method, a majority of patients, i.e., greater than 50 percent, requiring such long-term tube fed nutritional needs, will be provided with a specific distinct nutrition product. Thus, patients who are otherwise healthy and not elderly will be provided with a distinct product from cancer patients and elderly patients. In an embodiment, pediatric patients, patients suffering from digestive disorders, and patients suffering from cystic fibrosis will also each have a distinct nutritional formulation. Thus, healthcare practitioners will have the ability to provide specific patient populations with specific long-term nutrition products.

With respect to certain of the distinct formulas, by way of example, the maintenance product is designed to attempt to optimize metabolic status and stability in the population of long-term tube-fed patients designated herein as “maintenance”. These patients are typically “healthy,” stable, and normo-metabolic. The desire is to provide neither an excess nor a lack of calories and macro- and micro-nutrients. To the extent possible, the formula is designed to provide necessary macro- and micro-nutrients with certain differences to meet these patients' needs. The elderly long-term nutrition product is designed, in part, so as to optimize the glycaemic response. Hyperglycaemia can be a concern in elderly tube-fed patients. This can aggravate the chronic low-level inflammation typically present in elderly populations, which can lead to cardiovascular disease, diabetes and other diseases. With respect to the cancer long-term nutrition product, the long-term enteral nutrition is designed to address the effects of the cancer itself (inflammation, hypercatabolism) and the side effects of therapies such as radio- and chemo-therapy (mucositis).

By way of example and not limitation, examples of the long-term nutrition products that can be used in whole or in part for the methods of the present invention are as follows:

### Examples 1-2

### Elderly Long-Term Nutrition Product

### Example No. 1

### Elderly Product

| | | Embodiment | Embodiment |
|---|---|---|---|
| | | 1500 ml | per 100 ml |
| Calories | kcal | 1500 | 100.00 |
| Proteins | G | 60 | 4.00 |
| Whey protein | G | 36 | 2.40 |
| Soya protein isolate | G | 24 | 1.60 |
| Carbohydrates | G | 176 | 11.70 |
| Fructose | G | 16 | 1.1 |
| Maltodextrins | G | 86 | 5.8 |
| Starch | G | 57 | 3.8 |
| Carbohydrates from | G | 17 | 1.1 |
| other sources | | | |
| Fiber | G | 21 | 1.4 |
| Insoluble | % | 66 | 66 |
| Soluble | % | 34 | 34 |
| Lipids | G | 67 | 4.5 |
| SFA (includes MCT) | G | 20 | 1.3 |
| MUFA | G | 35 | 2.3 |
| PUFA | G | 10 | 0.68 |
| Linoleic acid (n-6) | G | 5.7 | 0.38 |
| α linolenic acid (n-3) | G | 1.1 | 0.07 |
| Ratio ω6/ω3 | | 3.1 | 3.1 |
| EPA | G | 0.45 | 0.03 |
| DHA | G | 0.30 | 0.02 |
| MCT | G | 10 | 0.67 |
| Minerals and | | | |
| Trace Elements | | | |
| Sodium | mg | 1185 | 79 |
| Potassium | mg | 3195 | 213 |
| Calcium) | mg | 1200 | 80 |
| Phosphorus | mg | 795 | 53 |
| Magnesium | mg | 345 | 23 |
| Chloride | mg | 1650 | 110 |
| Iron | mg | 12.3 | 0.82 |
| Zinc | mg | 15 | 1.0 |
| Copper | mg | 2.7 | 0.18 |
| Fluoride | mg | 1.2 | 0.08 |
| Chromium | μg | 225 | 15 |
| Molybdenum | μg | 195 | 13 |
| Selenium | μg | 100 | 6.7 |
| Manganese | mg | 4.0 | 0.27 |
| Iodine | μg | 150 | 10 |
| Vitamins | | | |
| Vitamin A total | IU | 4200 | 280 |
| Vitamin D | μg | 14.7 | 0.98 |
| Vitamin E | IU | 67.5 | 4.5 |
| Vitamin K | μg | 210 | 14.0 |
| Vitamin C | mg | 120 | 8.0 |
| Vitamin B1 | mg | 1.5 | 0.10 |
| (Thiamin) | | | |
| Vitamin B2 | mg | 1.95 | 0.13 |
| (Riboflavin) | | | |
| Vitamin B3-PP | mg | 15 | 1.00 |
| (Niacin) | | | |
| Vitamin B5 | mg | 8.0 | 0.53 |
| (Pantothenic acid) | | | |
| Vitamin B6 | mg | 2.25 | 0.15 |
| (Pyridoxine) | | | |
| Vitamin B8 | μg | 57 | 3.80 |
| (Biotin) | | | |
| Vitamin B9 | μg | 405 | 27 |
| (Folic Acid) | | | |
| Vitamin B12 | μg | 4.8 | 0.32 |
| Other | | | |
| Choline | mg | 795 | 53 |
| Carnitine | mg | 150 | 10 |
| Taurine | mg | 81 | 5.4 |
| Lycopene (tomato) | mg | 5.9 | 0.39 |
| Beta-carotene (carrot) | mg | 3.8 | 0.25 |
| Lutein (Spinach) | mg | 1.43 | 0.095 |

### Example No. 2

### Elderly Product

| | | Embodi- | | Embodi- |
|---|---|---|---|---|
| | | ment | RANGES for | ment |
| | | 1500 ml | 100 kcal | per 100 ml |
| Calories | kcal | 1500 | 0.8-1.3 kcal/ml | 100.00 |
| Proteins | g | 60 | 14-20% of total | 4.00 |
| | | | energy content | |
| | | | Any balanced | |
| | | | AA profile, | |
| | | | preferably >50% | |
| | | | of by weight of | |
| | | | proteins as whey | |
| | | | in partially | |
| | | | hydrolysed or | |
| | | | intact form | |
| Whey protein | g | 36 | | 2.40 |
| Soya protein | g | 24 | | 1.60 |
| isolate | | | | |
| Carbohydrates | g | 176 | 10-50% of total | 11.70 |
| | | | energy content | |
| | | | Selected to give | |
| | | | a lowered | |
| | | | glycemic | |
| | | | response, e.g. | |
| | | | glucose, sugar | |
| | | | alcohols | |
| | | | (sorbitol), | |
| | | | starches | |
| | | | (dextrins, | |
| | | | maltodextrins), | |
| | | | preferably should | |
| | | | not contain | |
| | | | sucrose | |
| Fructose | g | 16 | | 1.1 |
| Maltodextrins | g | 86 | | 5.8 |
| Starch | g | 57 | | 3.8 |
| Carbohydrates | g | 17 | | 1.1 |
| from other | | | | |
| sources | | | | |
| Fiber | g | 21 | 10-25 g/litre | 1.4 |
| Insoluble Fiber | % | 66 | | 66 |
| Soluble Fiber | % | 34 | at least 25% | 34 |
| | | | soluble | |
| | | | preferably 30- | |
| | | | 55% soluble | |
| Lipids | g | 67 | 30-45% of total | 4.5 |
| | | | energy content | |
| SFA (includes | g | 20 | saturated fats | 1.3 |
| MCT) | | | (not incl. MCT) <10% | |
| | | | of total energy | |
| | | | content or <1.11 | |
| | | | g/100 kcal | |
| MUFA | g | 35 | 40-60% of total | 2.32 |
| | | | lipids (by wt) | |
| PUFA | g | 10 | | 0.68 |
| Linoleic acid | g | 5.7 | | 0.38 |
| (n-6) | | | | |
| α linolenic | g | 1.1 | | 0.07 |
| acid (n-3) | | | | |
| Ratio ω6/ω3 | | 3.1 | 2 to 6 | 3.1 |
| EPA | g | 0.45 | 0.4-2% of total | 0.03 |
| | | | lipids by wt | |
| | | | (optional) | |
| DHA | g | 0.30 | | 0.02 |
| MCT | g | 10 | | 0.67 |
| Minerals and | | | | |
| Trace Elements | | | | |
| Sodium | mg | 1185 | | 79 |
| Potassium | mg | 3195 | | 213 |
| Calcium) | mg | 1200 | >35 | 80 |
| Phosphorus | mg | 795 | | 53 |
| Magnesium | mg | 345 | | 23 |
| Chloride | mg | 1650 | | 110 |
| Iron | mg | 12.3 | | 0.82 |
| Zinc | mg | 15 | | 1.0 |
| Copper | mg | 2.7 | | 0.18 |
| Fluoride | mg | 1.2 | | 0.08 |
| Chromium | μg | 225 | 8-18 | 15 |
| Molybdenum | μg | 195 | | 13 |
| Selenium | μg | 100.5 | | 6.7 |
| Manganese | mg | 4.0 | | 0.27 |
| Iodine | μg | 150 | | 10 |
| Vitamins | | | | |
| Vitamin A total | IU | 4200 | | 280 |
| Vitamin D | μg | 14.7 | >0.5 | 0.98 |
| Vitamin E | IU | 67.5 | | 4.50 |
| Vitamin K | μg | 210 | | 14.0 |
| Vitamin C | mg | 120 | | 8.0 |
| Vitamin B1 | mg | 1.5 | | 0.10 |
| (Thiamin) | | | | |
| Vitamin B2 | mg | 2.0 | | 0.13 |
| (Riboflavin) | | | | |
| Vitamin B3-PP | mg | 15 | | 1.00 |
| (Niacin) | | | | |
| Vitamin B5 | mg | 8.0 | | 0.53 |
| (Pantothenic | | | | |
| acid) | | | | |
| Vitamin B6 | mg | 2.3 | | 0.15 |
| (Pyridoxine) | | | | |
| Vitamin B8 | μg | 57 | | 3.80 |
| (Biotin) | | | | |
| Vitamin B9 | μg | 405 | | 27 |
| (Folic Acid) | | | | |
| Vitamin B12 | μg | 4.8 | | 0.32 |
| Other | | | | |
| Choline | mg | 795 | If present, >30 | 53 |
| | | | mg/100 kcal | |
| Carnitine | mg | 150 | If present >3 | 10 |
| | | | mg/100 kcal | |
| Taurine | mg | 81 | If present, >4 | 5.4 |
| | | | mg/100 kcal | |
| Lycopene | mg | 5.9 | >0.2 mg/100 kcal | 0.39 |
| (tomato) | | | | |
| Beta-carotene | mg | 3.8 | >0.1 mg/100 kcal | 0.25 |
| (carrot) | | | | |
| Lutein | mg | 1.43 | >0.05 mg/100 kcal | 0.095 |
| (Spinach) | | | | |

### Examples 3-4

### Long-Term Nutrition Maintenance

### Example No. 3

| | | Embodiment | Embodiment |
|---|---|---|---|
| | | Maintenance | per |
| | | per 1500 ml | per 100 ml |
| Calories | Kcal | 1875 | 125 |
| Protein | g | 62 | 4.1 |
| Ca Caseinate | g | 31 | 2.06 |
| Soya | g | 31 | 2.06 |
| Carbohydrates | g | 252 | 16.8 |
| Maltodextrins | g | 237 | 15.8 |
| Carbohydrates from | g | 15 | 1.0 |
| other sources | | | |
| Fiber | g | 23 | 1.52 |
| Insoluble | % | 66 | 66 |
| Soluble | % | 34 | 34 |
| Lipids | g | 72 | 4.8 |
| SFA | g | 11 | 0.73 |
| MUFA | g | 43 | 2.9 |
| PUFA | g | 11 | 0.73 |
| linoleic acid (n-6) | g | 8.4 | 0.56 |
| α linolenic acid (n-3) | g | 1.6 | 0.11 |
| Ratio ω6/ω3 | | 5.2 | 5.2 |
| Minerals and | | | |
| Trace Elements | | | |
| Sodium | mg | 2400 | 160 |
| Potassium | mg | 2445 | 163 |
| Calcium | mg | 1290 | 86 |
| Phosphorous | mg | 855 | 57 |
| Magnesium | mg | 405 | 27 |
| Chloride | mg | 3225 | 215 |
| Iron | mg | 18 | 1.2 |
| Zinc | mg | 12 | 0.78 |
| Copper | mg | 2 | 0.13 |
| Fluoride | mg | 1.4 | 0.09 |
| Chromium | μg | 105 | 7.0 |
| Molybdenum | μg | 98 | 6.5 |
| Selenium | μg | 81 | 5.4 |
| Manganese | mg | 4.4 | 0.29 |
| Iodine | μg | 165 | 11 |
| Vitamins | | | |
| Vitamin A total | IU | 4500 | 300 |
| Vitamin D | μg | 20 | 1.3 |
| Vitamin E | IU | 48 | 3.2 |
| Vitamin K | μg | 105 | 7.0 |
| Vitamin C | mg | 180 | 12.0 |
| Vitamin B1 | mg | 2.0 | 0.13 |
| (Thiamin) | | | |
| Vitamin B2 | mg | 1.7 | 0.11 |
| (Riboflavin) | | | |
| Vitamin B3-PP | mg | 23 | 1.50 |
| (Niacin) | | | |
| Vitamin B5 | mg | 9.5 | 0.63 |
| (Pantothenic acid) | | | |
| Vitamin B6 | mg | 2.3 | 0.15 |
| (Pyridoxine) | | | |
| Vitamin B8 | μg | 57 | 3.8 |
| (Biotin) | | | |
| Vitamin B9 | μg | 450 | 30 |
| (Folic Acid) | | | |
| Vitamin B12 | μg | 5.7 | 0.38 |
| Other | | | |
| Choline | mg | 810 | 54 |
| Taurine | mg | 81 | 5.4 |
| Carnitine | mg | 150 | 10 |
| Beta-carotene (carrot) | mg | 3.8 | 0.25 |
| Lycopene (tomato) | mg | 5.9 | 0.39 |

### Example No. 4

| | | Embodiment | | Embodi- |
|---|---|---|---|---|
| | | Maintenance | RANGE for | ment |
| | | per 1500 ml | 100 kcal | per 100 ml |
| Calories | kcal | 1875 | 0.8-1.4 kcal/ml | 125 |
| Protein | g | 62 | 10-18% of total | 4.1 |
| | | | energy content, intact | |
| | | | or partially | |
| | | | hydolysed | |
| Ca Caseinate | g | 31 | | 2.06 |
| Soya | g | 31 | | 2.06 |
| Carbohydrates | g | 252 | 40-65% of total | 16.8 |
| | | | energy content | |
| Maltodextrins | g | 237 | | 15.8 |
| Carbohydrates | g | 15 | | 1.0 |
| from other | | | | |
| sources | | | | |
| Fibers | g | 23 | >10 g/litre | 1.5 |
| Insoluble | % | 66 | | 66 |
| Soluble | % | 34 | | 34 |
| Lipids | g | 72 | 25-40% of total | 4.8 |
| | | | energy content | |
| SFA | g | 11 | saturated fats (not | 0.73 |
| | | | inc. MCT) <10% of | |
| | | | total energy content; | |
| | | | or <1.11 g/100 kcal | |
| MUFA | g | 43 | | 2.9 |
| PUFA | g | 11 | | 0.73 |
| Linoleic acid | g | 8.4 | 3-10% of total energy | 0.56 |
| (n-6) | | | content linoleic acid | |
| | | | or higher w6 | |
| | | | derivatives or | |
| | | | 0.33-1.11 g/100 kcal | |
| α linolenic | g | 1.6 | >0.6% of total energy | 0.11 |
| acid (n-3) | | | content or >0.06 | |
| | | | g/100 kcal | |
| Ratio ω6/ω3 | | 5.2 | 2-7 | 5.2 |
| Minerals and | | | | |
| Trace Elements | | | | |
| Sodium | mg | 2400 | 100-200 mg | 160 |
| Potassium | mg | 2445 | 25-250 mg | 163 |
| Calcium | mg | 1290 | At least 50 mg | 86 |
| | | | preferably 50-300 mg | |
| Phosphorus | mg | 855 | <150 mg | 57 |
| | | | preferably 40-80 mg | |
| Magnesium | mg | 405 | At least 15 mg | 27 |
| | | | preferably 15-35 mg | |
| Chlorides | mg | 3225 | At least 100 mg | 215 |
| | | | preferably 150-250 | |
| | | | mg | |
| Iron | mg | 18 | 0.4-1.5 mg | 1.2 |
| Zinc | mg | 12 | 0.4-2.0 mg | 0.78 |
| Copper | mg | 2 | 0.08-0.4 mg | 0.13 |
| Fluoride | mg | 1.4 | <0.15 mg | 0.09 |
| Chromium | μg | 105 | 2-10 μg | 7.0 |
| Molybdenum | μg | 98 | 2-14 μg | 6.5 |
| Selenium | μg | 81 | 3-9 μg | 5.4 |
| Manganese | mg | 4.4 | 0.1-0.4 mg | 0.29 |
| Iodine | μg | 165 | 7-15 μg | 11 |
| Vitamins | | | | |
| Vitamin A total | IU | 4500 | 100-500 IU | 300 |
| | | | inc. b-carotene | |
| Vitamin D | μg | 20 | 0.5-2.5 μg | 1.3 |
| Vitamin E | IU | 48 | 1.5-4 mg | 3.2 |
| | | | (2.2-6 IU) | |
| Vitamin K | μg | 105 | Greater than 4 | 7.0 |
| | | | μg/100 kcal | |
| | | | preferably 6-15 | |
| | | | μg/100 kcal | |
| Vitamin C | mg | 180 | Greater than 4 mg | 12.0 |
| Vitamin B1 | mg | 2.0 | Greater than 0.06 | 0.13 |
| (Thiamin) | | | mg/100 kcal | |
| | | | preferably 0.06-0.4 | |
| | | | mg/100 kcal) | |
| Vitamin B2 | mg | 1.7 | Greater than 0.07 mg | 0.11 |
| (Riboflavin) | | | | |
| Vitamin B3-PP | mg | 23 | 0.7-3.5 mg | 1.5 |
| (Niacin) | | | | |
| Vitamin B5 | mg | 9.5 | 0.2-2.0 mg | 0.63 |
| (Pantothenic | | | | |
| acid) | | | | |
| Vitamin B6 | mg | 2.3 | 0.1-0.7 mg | 0.15 |
| (Pyridoxine) | | | | |
| Vitamin B8 | μg | 57 | At least 1 μg | 3.8 |
| (Biotin) | | | | |
| Vitamin B9 | μg | 450 | At least 12 μg | 30 |
| (Folic Acid) | | | | |
| Vitamin B12 | μg | 5.7 | 0.1-1 μg | 0.38 |
| Other | | | | |
| Choline | mg | 810 | If present, >30 | 54 |
| | | | mg/100 kcal | |
| Taurine | mg | 81 | If present, >4 | 5.4 |
| | | | mg/100 kcal | |
| Carnitine | mg | 150 | If present >3 | 10 |
| | | | mg/100 kcal | |
| Beta-carotene | mg | 3.8 | >0.1 mg/100 kcal | 0.25 |
| (carrot) | | | | |
| Lycopene | mg | 5.9 | >0.2 mg/100 kcal | 0.39 |
| (tomato) | | | | |

### Examples 5-6

### Long-Term Nutrition Cancer

### Example No. 5

| | | Embodiment | |
|---|---|---|---|
| | | Formula | Embodiment |
| | | 1500 ml | per 100 ml |
| Calories | Kcal | 2310 | 154.00 |
| Protein | G | 129 | 8.6 |
| total)(includes | | | |
| free amino | | | |
| acids) | | | |
| Whey protein | G | 94 | 6.27 |
| hydrolysate | | | |
| added Leucine | G | 5 | 0.33 |
| added | G | 30 | 2.0 |
| Glutamine | | | |
| Carbohydrates | G | 273 | 18.2 |
| Maltodextrins | G | 254 | 16.9 |
| Starch | G | 15 | 1.0 |
| Carbohydrates | G | 4 | 0.27 |
| from other | | | |
| sources | | | |
| Fiber | | | |
| Lipids | G | 77 | 5.1 |
| SFA (includes | G | 34 | 2.3 |
| MCT) | | | |
| MUFA | G | 17 | 1.1 |
| PUFA | G | 20 | 1.3 |
| linoleic acid (n-6) | g | 14 | 0.92 |
| α linolenic acid (n-3) | g | 2.0 | 0.13 |
| Ratio ω6/ω3 | | 2.5 | 2.5 |
| EPA | g | 2.1 | 0.14 |
| DHA | g | 1.5 | 0.10 |
| Minerals and | | | |
| Trace Elements | | | |
| Sodium | mg | 2295 | 153.0 |
| Potassium | mg | 2700 | 180.0 |
| Calcium) | mg | 1140 | 76.0 |
| Phosphorous | mg | 1275 | 85.0 |
| Magnesium | mg | 405 | 27.0 |
| Chloride | mg | 2070 | 138.0 |
| Iron | mg | 14 | 0.93 |
| Zinc | mg | 28 | 1.9 |
| Copper | mg | 2.4 | 0.16 |
| Fluoride | mg | 1.95 | 0.13 |
| Chromium | μg | 115.5 | 7.7 |
| Molybdenum | μg | 285 | 19 |
| Selenium | μg | 135 | 9 |
| Manganese | mg | 5.4 | 0.36 |
| Iodine | μg | 300 | 20 |
| Vitamins | | | |
| Vitamin A total | IU | 8250 | 550 |
| Vitamin D | μg | 30 | 2.0 |
| Vitamin E | IU | 97.5 | 6.5 |
| Vitamin K | μg | 180 | 12 |
| Vitamin C | mg | 435 | 29 |
| Vitamin B1 | mg | 4.05 | 0.27 |
| (Thiamin) | | | |
| Vitamin B2 | mg | 4.35 | 0.29 |
| (Riboflavin) | | | |
| Vitamin B3-PP | mg | 40.5 | 2.7 |
| (Niacin) | | | |
| Vitamin B5 | mg | 16.5 | 1.1 |
| (Pantothenic acid) | | | |
| Vitamin B6 | mg | 5.25 | 0.35 |
| (Pyridoxine) | | | |
| Vitamin B8 | μg | 107 | 7.1 |
| (Biotin) | | | |
| Vitamin B9 | μg | 840 | 56 |
| (Folic Acid) | | | |
| Vitamin B12 | μg | 13.8 | 0.92 |

### Example No. 6

| | | Embodi- | | Embodi- |
|---|---|---|---|---|
| | | ment | RANGE for | ment |
| | | 1500 ml | 100 kcal | per 100 ml |
| Calories) | kcal | 2310 | preferably 1-2 | 154.00 |
| | | | kcal/ml | |
| Protein total | g | 129 | 15-30% of | 8.6 |
| (includes free | | | total energy | |
| amino acids) | | | content, | |
| | | | preferably 20- | |
| | | | 27% of total | |
| | | | energy content, | |
| | | | 14-25% of | |
| | | | total energy | |
| | | | content protein | |
| | | | (intact or | |
| | | | hydrolysed) | |
| | | | of which 50% | |
| | | | whey | |
| | | | 0.5-10% of the | |
| | | | energy of the | |
| | | | composition | |
| | | | comes from | |
| | | | glutamine | |
| | | | (free, bound or | |
| | | | short peptides), | |
| | | | preferably 3- | |
| | | | 7% | |
| Whey protein | g | 94 | | 6.27 |
| hydrolysate (g) | | | | |
| added Leucine | g | 5 | leucine: 0.8- | 0.33 |
| | | | 5% (free, | |
| | | | peptide or | |
| | | | bound) | |
| | | | preferably 1.5- | |
| | | | 3.5% of TEI | |
| | | | composition | |
| added | g | 30 | | 2.00 |
| Glutamine | | | | |
| Carbohydrate | g | 273 | 20-55% of | 18.2 |
| | | | total energy | |
| | | | content | |
| Maltodextrins | g | 254 | | 16.9 |
| Starch | | 15 | | 1.0 |
| Carbohydrates | | 4 | | 0.27 |
| from other | | | | |
| sources | | | | |
| Fiber | g | | Optional, but if | |
| | | | present, >10 | |
| | | | g/litre | |
| Lipids) | g | 77 | 25-40% of | 5.1 |
| | | | total energy | |
| | | | content, | |
| | | | preferably 30- | |
| | | | 35% | |
| SFA (includes | g | 34 | | 2.3 |
| MCT) | | | | |
| MUFA | g | 17 | | 1.1 |
| PUFA | g | 20 | | 1.3 |
| linoleic acid | g | 14 | | 0.92 |
| (n-6) | | | | |
| α linolenic | g | 2.0 | | 0.13 |
| acid (n-3) | | | | |
| Ratio ω6/ω3 | | 2.5 | | 2.5 |
| EPA | g | 2.1 | 1-5 g/day, or 1- | 0.14 |
| | | | 7% wt total | |
| | | | lipids | |
| DHA | g | 1.5 | | 0.10 |
| Mineral and | | | | |
| Trace Elements | | | | |
| Sodium | mg | 2295 | | 153.0 |
| Potassium) | mg | 2700 | | 180.0 |
| Calcium | mg | 1140 | | 76.0 |
| Phosphorus | mg | 1275 | | 85.0 |
| Magnesium | mg | 405 | At least | 27.0 |
| | | | 15 mg/100 | |
| | | | kcal, | |
| | | | preferably | |
| | | | 15-35 mg/100 | |
| | | | kcal | |
| Chloride | mg | 2070 | | 138.0 |
| Iron | mg | 14 | | 0.93 |
| Zinc | mg | 28 | 0.4-2.0 mg/ | 1.9 |
| | | | 100 kcal | |
| Copper | mg | 2.4 | | 0.16 |
| Fluoride | mg | 1.95 | | 0.13 |
| Chromium | μg | 115.5 | | 7.7 |
| Molybdenum | μg | 285 | | 19 |
| Selenium | μg | 135 | | 9 |
| Manganese | mg | 5.4 | | 0.36 |
| Iodine) | μg | 300 | | 20 |
| Vitamins | | | | |
| Vitamin A total | IU | 8250 | | 550 |
| Vitamin D | μg | 30 | | 2.0 |
| Vitamin E | IU | 97.5 | | 6.5 |
| Vitamin K | μg | 180 | | 12 |
| Vitamin C | mg | 435 | | 29 |
| Vitamin B1 | mg | 4.05 | | 0.27 |
| (Thiamin) | | | | |
| Vitamin B2 | mg | 4.35 | | 0.29 |
| (Riboflavin) | | | | |
| Vitamin B3-PP | mg | 40.5 | 0.7-3.5 mg/ | 2.7 |
| (Niacin) | | | 100 kcal | |
| Vitamin B5 | mg | 16.5 | 0.2-2.0 mg/ | 1.1 |
| (Pantothenic | | | 100 kcal | |
| acid) | | | | |
| Vitamin B6 | mg | 5.25 | 0.1-0.7 mg/ | 0.35 |
| (Pyridoxine) | | | 100 kcal | |
| Vitamin B8 | μg | 107 | At least 1 μg/ | 7.1 |
| (Biotin) | | | 100 kcal | |
| Vitamin B9 | μg | 840 | At least 12 μg/ | 56 |
| (Folic Acid) | | | 100 kcal | |
| Vitamin B12 | μg | 13.8 | 0.1-1 μg/ | 0.92 |
| | | | 100 kcal | |

In any of the above examples, the long-term nutrition product will be marketed to or made available to the specific patient population for providing long-term enteral nutrition. These products can be used to develop and/or maintain a business.

By providing such a business, necessary health needs can be met while providing revenue to the business or businesses providing these services and products.

The present invention allows for the development of a business strategy based on the long-term enteral nutrition market. This strategy can include product branding, which will allow, in part, the product to be specifically positioned. In addition, specific markets can be targeted, for example, out-of-hospital/home care market. By providing necessary nutrition products and equipment, a differentiated product offering can be achieved in terms of services offered and products offered. Thus, by offering a complete solution to the challenge of long-term enteral feeding in an out-of-hospital environment, a new business method can be implemented.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for providing long-term tube fed nutrition to at least a majority of patients requiring same comprising the steps of:
providing a line of tube fed nutrition products that are designed to provide long-term nutritional requirements of at least certain patient populations, the patient populations selected from the group consisting of: elderly, maintenance, cancer, digestive pathologies, pediatric, stroke, coma, respiratory conditions and cystic fibrosis;
providing for each of the certain patient populations the necessary nutrients to support the patient long-term; and
marketing each of the product lines to the target patient population.

2. The method of claim 1 wherein the patient receives the product at home.

3. The method of claim 1 wherein the nutrition product is provided to the patient with at least a feed tube selected from the group consisting of nasogastric and percutaneous endoscopic gastrostomy.

4. The method of claim 1 wherein the long-term nutrition products are provided in a bag.

5. The method of claim 1 wherein the long-term nutrition product is sold as a package comprising disposable items and instructions with respect to the product.

6. The method of claim 5 wherein the package is delivered to the home of the patient.

7. A method of providing long-term nutrition to specific patient populations comprising the steps of:
providing at least three distinct long-term tube fed nutrition products each designed for a distinct specific patient population; and
marketing each of the long-term nutrition products to the specific patient population for long-term tube fed nutrition.

8. The method of claim 7 wherein the specific patient population is selected from the group consisting of: maintenance; elderly; cancer; digestive pathologies; and pediatric.

9. The method of claim 7 wherein the nutrition products are designed to mimic 5/8 a day.

10. The method of claim 7 wherein the patient receives the nutrition product at home.

11. The method of claim 7 wherein the nutrition product is provided to the patient with at least a feed tube.

12. The method of claim 7 wherein the nutrition product is sold as a package with necessary disposables and instructions as to how to use the nutrition product and disposables.

13. The method of claim 12 wherein the package is delivered to a home of the patient.

14. A method of marketing long-term nutrition to distinct patient populations at home comprising the steps of:
marketing to at least three distinct patient populations three distinct long-term tube fed nutrition products with equipment and instructions to allow the patient to receive long-term tube fed enteral nutrition at home, the patient populations comprising at least maintenance and elderly.

15. The method of claim 14 wherein the patient populations additionally comprise pediatric, digestive pathologies, and cancer.

16. The method of claim 14 wherein the nutrition product mimics 5/8 a day.

17. The method of claim 14 wherein the nutrition product is marketed to a healthcare practitioner.

18. A method of providing long-term tube fed nutrition to at least three distinct patient populations comprising the steps of:
providing three distinct tube fed nutrition products designed to provide long-term nutrition;
providing equipment to allow the patient to receive the long-term nutrition product at home; and
providing training to the patient on how to use the equipment as to receive long-term nutrition at home.

19. The method of claim 18 wherein the equipment comprises at least one product selected from the group consisting of a nasogastric tube, a percutaneous endoscopic gastrostomy tube, a flow regulator, and a pump.

20. The method of claim 18 wherein the training includes an individual that meets with the patient.

21. The method of claim 18 wherein the patient population is selected from the group consisting of: maintenance, elderly, cancer, digestive pathologies, and pediatric.

22. The method of claim 18 wherein the nutrition product mimics 5/8 a day.

23. The method of claim 18 comprising the step of providing the patient with the ability to provide feedback to a prescriber of the nutrition product.

24. The method of claim 18 comprising the steps of delivering the products to a home of the patient.

25. The method of claim 24 wherein the nutrition product is delivered by a healthcare professional.

26. The method of claim 25 wherein the healthcare professional is selected from the group consisting of: doctor, nurse, and dietitian.

27. A method of providing enteral nutrition products comprising the steps of offering for sale standard enteral nutrition products and long-term enteral nutrition products.

28. The method of claim 27 wherein the long-term nutrition products comprise at least two distinct nutrition products designed for distinct patient populations.

29. The method of claim 27 including the step of providing feed tubes.

30. The method of claim 27 including the step of providing means for controlling the flow of enteral feeds.

31. The method of claim 27 including the step of providing the services of a healthcare practitioner.

32. The method of claim 27 including the step of working with a third party selected from the group consisting of hospital, nursing home, and home healthcare center to provide enteral nutrition to a patient.

33. The method of claim 27 including the step of creating and maintaining a database including data on patients using the enteral nutrition products.

34. The method of claim 27 including the step of training healthcare practitioners to use the enteral product.

35. A method for generating revenue comprising the step of basing at least a portion of a business on providing long-term tube-fed nutrition to specific patient populations including providing at least one component to be used with the enteral nutrition selected from the group consisting of: percutaneous endoscopic gastrostomy tube; nasogastric feed tube; instructions; training; and means for controlling the flow of enteral feed.

36. The method of claim 35 comprising the step of providing the patient with the ability to provide feedback to a prescriber of the nutrition product.

37. The method of claim 35 wherein the long-term nutrition products comprise at least two distinct nutrition products designed for distinct patient populations.

38. The method of claim 35 including the step of providing the services of a healthcare practitioner.

39. The method of claim 35 including the step of working with a third party selected from the group consisting of hospital, nursing home, and home healthcare center to provide enteral nutrition to a patient.

40. The method of claim 35 including the step of creating and maintaining a database including data on patients using the enteral nutrition products.

41. The method of claim 35 including the step of training healthcare practitioners to use the enteral product.
